(19) United States
(12) Patent Application Publication
(10) Pub. No.: US 2010/0152686 A1
(43) Pub. Date: Jun. 17, 2010

(54) Collecting System Suitable for Collecting and Disposing of Bodily Waste Materials

(76) Inventors: Gerard Ryder, New Ross (IE); Anne Houston, New Ross (IE)

Correspondence Address: THE WEBB LAW FIRM, P.C., 700 KOPPERS BUILDING, 436 SEVENTH AVENUE, PITTSBURGH, PA, 15219, US

(21) Appl. No.: 12/520,305

(22) PCT Filed: Dec. 20, 2007
(86) PCT No.: PCT/IE2007/000125

§ 371 Date: Jan. 12, 2010

(30) Foreign Application Priority Data
Dec. 21, 2006 (IE) S2006/0949

(51) Int. Cl. A61F 5/445

(52) U.S. Cl. 604/332

(57) ABSTRACT

The present invention relates to a collecting system with a collecting bag for collecting bodily waste materials, a discharge outlet and a disposable receptacle for receiving bodily waste materials from a discharge outlet of the collecting bag. The receptacle has an inlet and a securing means for securing it to the collecting bag. The disposable receptacle and collecting bag are movable between a collecting state where the collecting bag is arranged for collecting bodily waste materials and there is substantially no communication of bodily waste materials into the disposable receptacle, and a discharge state where bodily waste materials can be discharged into the disposable receptacle.

### FIELD OF THE INVENTION

The present invention relates to a collecting system for collecting bodily waste and further comprises a means for disposing of the bodily waste materials. In particular the present invention concerns disposal of bodily waste materials which are collected from an artificial excretory opening.

Surgical construction of an artificial excretory opening generally takes place as an ostomy procedure. In particular, the present invention is useful to ostomy patients such as those who have had an ileostomy or colostomy

### BACKGROUND TO THE INVENTION

Following surgery, patients who have had a surgical construction of an artificial excretory opening such as ileostomy or colostomy patients use ileostomy/colostomy bags to collect bodily waste materials. These bodily waste materials include gases, liquids and solids. The waste material may be semi-solid faecal waste. It is desirable in any event to discharge the collected materials with minimal handling from the user.

These ileostomy/colostomy bags are intended for multiple use purposes and generally the surgical patient finds that they have to empty the collection bag many times during the day. A given collection bag is thus fitted with waste discharge outlet through which the waste materials collected from the artificial excretory opening can be discharged. It is not unusual to empty these bags between six and ten times in any given day. A collection bag will typically be worn for a number of consecutive days before being replaced with a new bag. Given the nature of the materials which they collect, such collection bags are generally made of plastics materials.

During the emptying procedure the patient or bag wearer generally has to squat down beside a toilet to allow direct emptying of the collection bag into the toilet. This is due in part to the typical positioning of the artificial excretory opening on the body. Often times the opening on the body is provided at or about the stomach area and often to one side of the body. It is not usually possible therefore for the bag wearer to easily directly empty the collected bodily waste material into a toilet if they are in a seated position on the toilet. A position to one side of the toilet is thus required.

Another option which some bag wearers use is to empty the collected bodily waste materials into a receptacle such as a plastic jug. The receptacle may then in turn be emptied into a toilet. Even with this method of emptying the bag it is difficult to use a toilet in a conventional fashion as if seated the user will likely have to stand up to empty the receptacle.

Generally therefore conventional procedures for emptying such collection bags are inconvenient.

This problem of inconvenience for the user has been identified. The inventors are aware of a number of proposals which seek to help a bag wearer dispose of bodily waste materials from the bag.

For example, U.S. Pat. No. 4,387,713 describes a disposable discharge collector for a stoma pouch. The discharge collector which includes a plastics film body bag. A liquid tight seal is provided on the collector as are wipers for wiping the collection bag after discharge. As described in that document, the stoma pouch is rolled up and secured by a spring clip during all times other than when the stoma pouch is being emptied. The emptying process is complex, involving the release of the pin while achieving the correct positioning of the wipers relative to the discharge outlet, the manual handling of both the discharge collector and the collection bag, a wiping action, and a subsequent sealing of the locking mechanism. The discharge collector of this patent is considered by the present inventors to be quite difficult to use, requiring a lot of manual dexterity, and it is believed that undesired spillage would occur.

Another arrangement is disclosed in German patent publication DE 44 18 789 A1. This invention concerns a closable container for the admission of fluids for example urine or faeces. The container is made from flexible foils welded together. A series of adhesive strips are employed, a first pair of adhesive strips of attaching to the foil container and a second series of adhesive strips for attaching to a second container. A more complex arrangement with up to six adhesive strips is also employed. Separation of certain of the adhesive strips by cutting is also disclosed. The foil bag must be put in place for collection of material from an unspecified second container. There is no mention of ostomy bags and the like and the various configurations again require manual dexterity in order to allow their use without spillage of materials.

U.S. Pat. No. 5,951,532, describes a collecting bag system for human body wastes comprising a collecting bag with an inlet opening surrounded by connecting elements for connection to a body orifice. One aspect of the invention includes an extension drain to the bag which allows material making its way into the collecting bag to be continuously drained off into a large collecting bag. The invention is aimed as patients that have mobility restrictions and/or are confined to bed as evidenced by the rather cumbersome arrangement. Indeed the arrangement itself is described as a bedside arrangement for the collection of fluids. The apparatus described is unsuitable for patients that are mobile.

U.S. Pat. No. 3,825,005 describes a reusable, resealable, ileostomy or colostomy bag which may be emptied without removing the bag from the stoma of the user and is intended for use by a bedridden person. The receptacle has a complicated construction with the necessity for ribs integral with the panel to provide a seal. Again a continuous drainage arrangement is shown where a drain leads down to an accumulator which collects the continuously draining material from the bag. Again this arrangement is not suited for use by mobile collection bag wearers.

U.S. Pat. No. 3,841,332 relates to an enterostomy drainage bag having front and rear walls of flexible moisture-proof material forming a fluid collection chamber therebetween. The bag is intended for continuous drainage use for a person who remains in one place being primarily aimed at patients confined to bed or for night time use. A flexible tube is required to drain the bag which can be connected to a larger remote collection container. This bulky equipment is not considered portable.

UK patent application No. GB 2,258,399 and U.S. Pat. No. 4,285,076 each describe a fixed plumbed device for emptying and cleaning a collection bag for excreta, while U.S. Pat. No. 5,671,485 discloses a device that can be fitted onto a toilet seat to support a colostomy bag. Other ostomy pouches, which may be disposed through toilets, are described in Canadian patent application No. 1,320,324 and U.S. Pat. No. 5,769,831, U.S. Pat. No. 4,930,942 describes a wrapping or enclosure into which flexible soft objects such as ostomy pouches can be placed. The material of the wrapping or enclosure becomes slimy on contact with water in the toilet bowl thus allowing for flushing. An inner and outer bag arrangement is described in the German patent application DE 19,519,069,

U.S. Pat. No. 5,938,647 to Welland Medical Limited discloses a biodegradable flushable ostomy bag liner. The liner is designed to be used in a two-bag ostomy bag system. The liner is adapted to fit into the outer bag and collect the waste material. When full the liner can be removed from the outer bag and disposed of by flushing in a toilet. The outer bag may have to be disposed of separately for example in a dustbin.

One of the drawbacks of this system is that the end user has to replace both the inner and the outer bag when the inner bag is full. This may prove difficult for people with a lack of dexterity.

One commercial toilet flushable pouch (similar to the device described in U.S. Pat. No. 5,938,647) is available from Welland Medical Limited in the UK. Their product sold under the trademark. Flair Xtra™ is an inner liner pouch which is designed to fit within the bag worn by the user. The pouch is designed and arranged to collect the bodily waste materials and provide a barrier between those materials and the bag. None of the bodily waste materials therefore comes into direct contact with the inner of the bag. Additionally, the bodily waste materials can be removed by removing the inner pouch without contamination of the bag.

This means however that the inner pouch must be of a relatively complex design as it must be adapted to fit to the artificial opening of the body in the same way as the collection bag. This is necessary in order to avoid contamination of the collection bag with bodily waste material.

In general the containers and methods of disposal of the prior art require a high level of dexterity and are not convenient for the end user and are generally difficult to carry around on the end user's person.

International (PCT) application no. PCT/IE2006/000064 to the present inventors discloses a disposal receptacle which is very convenient for discrete use and is easily portable. The inventors have now devised further improvements to such disposal receptacles.

### SUMMARY OF THE INVENTION

The present invention describes a collecting system as set out in the appended claims. Furthermore, the invention relates to a method of disposing of collected bodily waste materials.

The collecting system of the present invention includes a collecting bag for collecting bodily waste materials, the collecting bag comprising a discharge outlet; a disposable receptacle for receiving bodily waste materials from a discharge outlet of the collecting bag, the receptacle comprising an inlet opening formed on the body; and a securing means for securing the receptacle to the collecting bag. The disposable receptacle and collecting bag are movable between a collecting state where the collecting bag is arranged for collecting bodily waste materials and there is no communication of bodily waste materials into the disposable receptacle; and a discharge state, where bodily waste materials can be discharged into the disposable receptacle. In both states the discharge outlet of the collection bag is connected to the inlet opening of the disposable receptacle. The disposable receptacle may be in a compact configuration when it (and the collecting bag) is in the collecting state.

The advantages of the present invention are manifold. Firstly, the collection bag and the disposal receptacle can be put in place ever before the collection bag is employed to collect materials. For example the user can pre-assemble the collection bag and the receptacle before the collection bag is put in place for use.

When it comes to emptying the collection bag, because the disposable receptacle is already pre-attached in a bodily waste material receiving configuration, release of the material into the disposable receptacle is very easily and simply achieved. In most cases, it involves removing a seal or other such restriction which has been applied to the collection bag to stop bodily waste materials being inadvertently discharged therefrom.

This restriction may be formed by rolling the receptacle for example the restriction may be formed by folding the receptacle. Most preferably the restriction is formed by rolling both the receptacle and a drain of the collecting bag in the same roll. In the present description rolling includes flat rolls such as those formed by folding.

A drain of the collecting bag may be a discharge neck or spout on the bag that forms a discharge conduit to the discharge outlet.

For example where a collection bag is closed by rolling it about itself, the disposable receptacle can be incorporated into such a roll, for example by rolling both up in the same roll. This means that the disposable receptacle can be very neatly attached to the collection bag. Furthermore when it is desired to empty the collection bag, the normal opening action of the discharge outlet of the collection bag is achieved in the routine manner e.g. by unrolling.

For example the restriction may be removed by unrolling the rolled receptacle. Generally opening out the disposable receptacle, for example from a compact configuration, removes the restriction on the discharge outlet. It may be necessary to open out both the disposable receptacle and a drain of the collecting bag to remove the restriction on the discharge outlet if the drain of the collecting bag and the disposable receptacle have been rolled in the same roll. Once the restriction has been removed and the folded receptacle has been unfolded, the collecting system has been expanded and is considered to be in the discharge state, where bodily waste may be communicated into the waste receiver chamber of the receptacle. In the expanded state discharge into the disposable receptacle desirably automatically occurs without the need for further action on the part of the user.

The receptacle will generally receive the bodily waste through an inlet opening. The receptacles inlet opening is preferably situated proximate to, or at the top end thereof. The receptacle body is adapted to receive within the inlet opening the bodily waste discharge outlet of the collection bag for example by insertion of a drain of the collection bag through the inlet opening.

The receptacle may comprise a bodily waste material-receiving chamber and the securing means holds the bodily waste material-receiving chamber in position for discharge of the bodily waste materials into the bodily waste material-receiving chamber from the collection bag. The bodily waste will be communicated through the discharge outlet of the collection bag.

The collecting system may comprise a receptacle which has at least one wiper such as a wiper pad arranged for wiping the discharge outlet of the collection bag and where a drain is present, desirably the drain is wiped along a sufficient length to remove any remaining waste material. Preferably the receptacle will comprise two opposing wipers for example opposing wiper pads on opposing sides of the receptacle, so that both sides of the discharge outlet can be wiped when the disposable receptacle is being removed. The wiper pad(s) may serve a number of different functions; such as to hold open an insertion portion in the disposable receptacle when it is attached to the collecting bag. For example where a self sealing mouth is provided on the disposable receptacle it is desirable that the insertion portion is sufficiently wide to allow the bodily waste material to discharge into the disposable receptacle.

The wipes or wiper pads will also serve to clean the drain of the collecting bag. Where pads are employed they can be made from any adsorbent material. This allows the pads to absorb any residual bodily waste matter from the outlet of the collection bag, for example residual material present after emptying. Since the wipes will generally be visually distinct from the receptacle bag material they will also function as a visual marker identifying the correct position for the outlet of the collection bag relative to the receptacle. The drain of the collecting bag is desirably placed between opposing pads in the discharge state.

The wiper pads usually extend between 1 cm to 5 cm into the inlet opening of the bodily waste receiving chamber, most preferably the pads will extend between 1-2 cm into the inlet opening of the bodily waste receiving chamber. The wipes may alternatively extend about substantially the entire periphery of the disposable receptacle.

With the additional wiping action of the present invention the discharge outlet of the collection bag can be cleaned. The new disposable receptacle can then be attached and it is pre-applied before the collection bag is used to collect further materials. It will be appreciated that in doing so, the manual dexterity required of previous systems is eliminated and the convenience for the user is evident. There is no necessity to align the two individual containers, one of which is at least partially filled, and deal with any inadvertent spillages which occur due to relative movement of the collection bag and the collecting receptacle. Release of the material from the collection bag is the step most likely to result in spillage.

To further facilitate patients with poor dexterity, finger grip portions may be applied to the collecting system. The finger grip portions allow the disposable receptacle bag to be opened easily. It is usual to have at least one finger grip portion which projects away from the receptacle body about the inlet opening. Most preferably the receptacle will comprise two finger grip portions both of which will project away from the receptacle body and positioned about the inlet opening. It is usual that the finger gripping portions are distinguishable from the rest of the body to assist end users who have poor eye sight to locate it. For example, the finger gripping portion may be a different colour to the rest of the body. Additionally or alternatively the finger gripping portions may be a different texture to the rest of the body.

The receptacle may be designed to possess a guard portion that is attached to the receptacle adjacent the inlet opening and which is arranged to project from the receptacle to a position alongside the collection bag. The guard portions will prevent splashing while the collection bag is being emptied.

The receptacle of the collecting system may have a receptacle body that is self-securing to the collection bag at a plurality of positions for example at a series of discrete position or along the entire receptacle.

Desirably the receptacle of the collecting system is toilet-flushable. Usually the design of the receptacle will lend itself to the flushability of the disposable receptacle bag, wherein the elongate body of the receptacle becomes narrower in a direction from the top end thereof towards the base end thereof.

The disposable receptacle will be made from adaptable material which allows the bag to be collapsed from an expanded state for use to receive the discharged waste materials from the collection bag, to a collapsed state where the bag is rolled, for example folded flat, to a convenient size for storage.

To allow the disposable receptacle to be comfortably worn by the patient the collapsed state of the receptacle is usually a substantially flat condition thereof.

Desirably the receptacle is formed from a plastics material folded upon itself and sealed at opposing sides to form a bag. The plastics material will be selected to disintegrate in water. This means the receptacle remains toilet-flushable. The plastics material may be any suitable plastics material for example a polyvinyl alcohol material. This makes a very simple yet effective construction.

The securing means may be formed utilising a double-sided adhesive tape. Again this allows a simple construction and is an effective method of adhering to a receptacle. For example one side of the double-sided adhesive tape may be attached to the receptacle and the other side will be provided with a protective layer which is removable for adhesively securing the receptacle to the collection bag. Differing adhesive strengths may thus be provided on the opposing sides of the tape—for example permanent bonding to the receptacle and temporary bonding to the collection bag.

The collecting system may further comprise a fastener. The fastener is used to hold the system in a collecting state. Preferably the fastener is a clip such as a j-clip. Alternatively the fastener may be an adhesive fastener for example a non-permanent adhesive or a mechanical fastener such as a hook and loop fastener. In many cases the fastener will be adapted to cooperate with a fastener arrangement already on the collection bag.

The receptacle further comprises a closure for its inlet opening. This closure will allow the disposable receptacle to be sealed once it has been removed from the collecting bag. Sealing the disposable receptacle will prevent undesired spillage of the waste material and optionally allows the receptacle to be disposed into a waste bin rather then flushed down the toilet.

The collecting system may be used for collecting and disposing of bodily waste materials. The mode of operation may include attaching a disposable receptacle with an inlet, to a collecting bag with a bodily waste discharge outlet, so that the inlet opening receives the bodily waste discharge outlet of the collection bag. Subsequently arranging the disposable receptacle and collecting bag into a collecting state where the collecting bag is arranged for collecting and there is no communication of waste into the disposable receptacle. Once in the collecting state a restriction is formed at the drain of the collecting bag and bodily waste may be collected into the collecting bag. Once the collecting is complete the disposable receptacle may be moved into a discharge state wherein the bodily waste material can be discharged from the collecting bag into the disposable receptacle. The bodily waste contents of the collecting bag may then be discharged into the receptacle and optionally the receptacle and any contents thereof may be disposed of.

The present invention also relates to a disposable receptacle comprising:
(i) a bag body having front and rear walls and a mouth;
(ii) securing means in the form of a double-sided adhesive tape one side of which is attached to the receptacle and the other side is provided with a protective layer which is removable for adhesively securing the receptacle to the collection bag.

Again this is a simple yet highly effective construction which provides ease of use and ease of disposal. To allow toilet disposal the bag body may be constructed of a plastics material which disintegrates in water for example a polyvinyl alcohol.

It is also very simple to form a receptacle of the invention. The receptacle can be formed by folding a plastics material (such as a film) upon itself and sealing opposing sides to form a bag.

The disposable receptacle component of the collecting system is not restricted to the embodiment described above. Alternatively the disposable receptacle element of the collecting system may have a bag body having front and rear walls, a mouth and a front securing flap integrally formed with the front wall of the bag and arranged to project above the mouth of the bag. There may also be a rear securing flap integrally formed with the rear wall of the bag and arranged to project above the mouth of the bag. The bag may further comprise a securing means on each of the flaps for securing the flaps to opposing sides of a collecting bag. In use, the flaps secure the receptacle to a collecting bag.

Yet another embodiment of the disposable receptacle component of the collecting system includes a disposable receptacle comprising a bag body having front and rear walls, a mouth and opposing securing flaps mounted on opposing sides of the bag body for securing the bag body to opposing sides of a collecting bag having a collapsible drain with a discharge outlet, in a configuration where the flaps do not secure the mouth of the bag body to the drain.

In any of the embodiments described the flaps of the disposable receptacle may be integrally formed with the bag.

Any of the disposable receptacle component embodiments as described above may further comprise at least one pad arranged to abut the drain of the collecting bag.

Preferably the disposable receptacle component will comprise opposing pads for abutting opposing sides of the drain of the collecting bag. These pad(s) may further act as wiper pad(s) for wiping the drain of the collecting bag. The pad(s) usually will be made from absorbent material so that the pad(s) will be absorbent pad(s).

All the various different embodiments of the disposable receptacle described above are suitable for use in a collecting system in accordance with this invention. Further more all these disposable receptacles as described herein are capable of being moved into a folded configuration for attachment to a collecting bag. The folded configuration allows any of the disposable receptacles to be comfortably worn by the patient.

The present invention may exist in an assembled form. The assembly comprising; a collecting bag for collecting bodily waste materials, the collecting bag comprising a discharge outlet; a disposable receptacle for receiving bodily waste materials from a discharge outlet of the collecting bag, the receptacle comprising an inlet opening formed on the body; the disposable receptacle being attached to the collecting bag so as to travel with and on the collection bag, the disposable receptacle being in a compact configuration. The compact configuration may be wherein the disposable receptacle is folded. It is usual that the folded configuration includes at least one longitudinal fold and at least one transverse fold.

### BRIEF DESCRIPTION OF THE DRAWINGS

FIG. 1 shows a front elevational view of the collecting system of the invention including a collecting bag and a first embodiment of a disposable receptacle component.

FIG. 2a shows a front elevational view of a stage in the method for moving the collecting system of FIG. 1 into the collecting state.

FIG. 2b shows a front elevational view of a subsequent stage in the method for moving the collecting system of FIG. 1 into the collecting state.

FIG. 3 shows an elevational view of the collecting system in the collecting state.

FIG. 4 shows a perspective view of a second embodiment of a disposable receptacle component of a collecting system.

FIG. 5 shows a perspective view of the disposable receptacle of the type shown in FIG. 4 further including wiper pads.

FIG. 6 shows a perspective view of a collecting system according to the invention with the receptacle of FIG. 4 attached partially to the collecting bag.

FIG. 7 shows a plan view of the collection system of FIG. 6 in a pre-use or discharge state.

FIG. 8 shows a plan view of the collection system of FIG. 7 and further illustrates lateral folding of the receptacle to put it in a compact configuration.

FIG. 9 shows a front elevational view of the collection system of FIG. 8 with further transverse folding to form a restriction for the discharge outlet of the collecting bag.

FIG. 10 shows a front elevational view of the collecting system of FIG. 6 in the collecting state.

FIG. 11 shows a cross sectional view through the collection system of FIG. 10 after bodily waste has been discharged from the collection bag into the disposable receptacle.

FIG. 12 is a perspective view of a disposable receptacle of the invention, having a further closure or sealing means.

FIG. 13 shows a front elevational view of a further collecting system of the invention including a collecting bag and a further embodiment of a disposable receptacle.

FIG. 14 shows a front elevational view of a stage in the method for moving the collecting system of FIG. 13 into the collecting state.

FIG. 15 shows an elevational view of the collecting system of FIG. 13 in the collecting state.

### DETAILED DESCRIPTION OF THE DRAWINGS

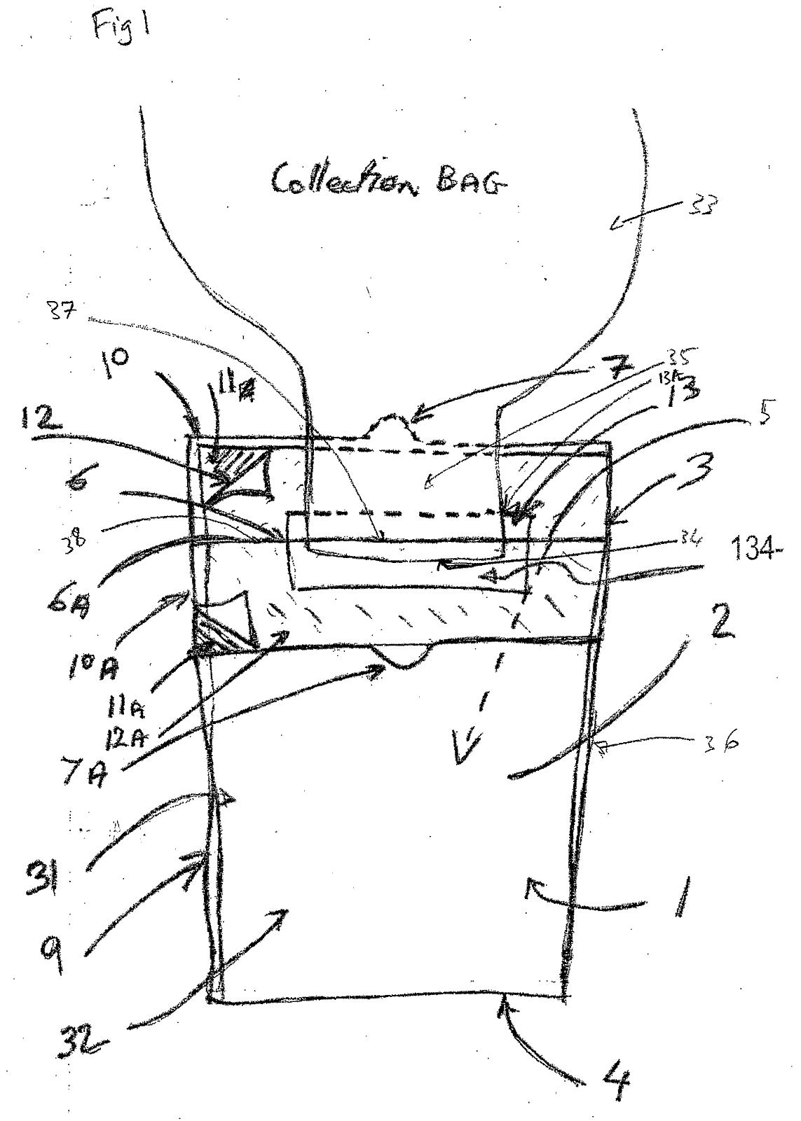

FIG. 1 shows a collecting system comprising a collecting bag 33 for collecting bodily waste, the collecting bag comprising a discharge outlet 34. The collecting system further comprises a disposable receptacle 1 for receiving bodily waste materials from a discharge outlet 34 of the collecting bag 33. The receptacle 1 has a mouth 38 that is located at the top end thereof. In the embodiment the mouth runs the width of the disposable receptacle and provides an opening into the disposable receptacle. It will be appreciated that the mouth could be shorter than the width of the receptacle. The receptacle further comprises an inlet opening 6 formed on the receptacle body, proximate to, or at the top end thereof. In the embodiment the inlet opening 6 is at the top end 3 of the elongate body 2 but of course could be alternatively located for example located proximate thereto. The body of the receptacle is adapted to receive within the inlet opening the bodily waste discharge outlet 34 of the collection bag 33. In the embodiment the discharge outlet 34 is on a collapsible drain 35 so as to allow bodily waste materials within the collection bag to be discharged into the bodily waste material-receiving chamber. The drain 35 is a narrow neck of material extending from the main collection chamber of the bag. Preferably, the inlet opening 6 is configured so that the drain 35 and outlet 34 of the collection bag can sufficiently extend into the waste material-receiving chamber 5 to reduce the risk of spillage.

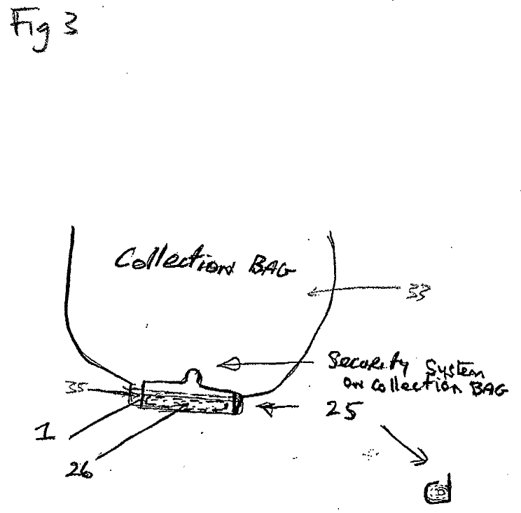

The disposable receptacle and collecting bag is movable between a collecting state as shown in FIG. 3 where the collecting bag is arranged for collecting and there is no communication of waste into the disposable receptacle and a discharge state as shown in FIG. 1 where material can be discharged. In both states the discharge outlet is connected to the inlet opening of the disposable receptacle. FIG. 1 shows the assembly comprising the collecting bag for collecting bodily waste materials, the collecting bag comprising a discharge outlet 34; and the disposable receptacle 1. The disposable receptacle is attached to the collecting bag so as to travel with the collection bag. In this configuration it is desirable that the disposable receptacle is in a compact configuration.

The receptacle has an elongate body 2 with a top end 3, a base end 4, an intermediate portion 36 between the top end 3 and the base end 4, and is formed with an internal, bodily waste material-receiving chamber 5.

Desirably the receptacle has opposing side walls (9a,9b) and also opposing front and rear walls (32,31). The opposing side walls 9a and 9b may be of different heights and maybe formed by sealing the edges of the front 32 and rear 31 walls. The edges of walls 31 and 32 may be sealed by any suitable means. One suitable sealing means is heat sealing. Alternatively the opposing walls may be formed by a seamless bag making process.

The elongate body 2 and waste material-receiving chamber 5 are of suitable dimensions so that they can receive waste material from a bodily waste discharge collection bag (such as a colostomy or an ostomy collection bag) without overflowing. For example, the internal dimensions of the waste material-receiving chamber 5 are adequate to receive the volume of waste material discharged from the collection bag. The disposable receptacle of the present invention can be manufactured in a number of sizes. For example smaller sized disposable bags 1 may be suitable for use by children or persons with a requirement for small discharge collection bags. Whereas an adult user may require a larger bag to a disposable receptacle as the volume of waste that their discharge collection bag holds will be greater. Typical dimensions of the disposable receptacle are in the region of 36 cm×16 cm to 20 cm×6 cm, such as 30 cm×12 cm to 24 cm×8 cm, for example 28 cm×10 cm to 26 cm×10 cm. Disposable receptacles of the present invention may be marked, for example, colour-coded for size to indicate the maximum volume the waste material-receiving chamber 5 can accommodate.

Disposable receptacles of the present invention may have an indication such as a colour coding or the like to indicate the intended end user size etc.

The length of the receptacle will vary depending on the size of the end user. For example a child would require a smaller length compared to an adult. Typically, the length 30 of the receptacle would be in the region of 34 cm to 20 cm, such as 32 cm to 24 cm, for example 30 cm to 26 cm. The product may be colour coded to indicate its length.

The receptacle can be seen to further comprise a securing means in the form of adhesive strips 11,11A for holding the bodily waste material-receiving chamber 5 in position for discharge of the bodily waste materials into the bodily waste material-receiving chamber 5 from the collection bag 33. Any suitable securing mean may be employed.

FIG. 1 shows opposing flap portions 10 and 10A each integrally formed with opposing sides of the bag. On the respective flap portions 10;10A are located securing means in the form of adhesive strips 11 and 11A respectively. The flap portions 10;10A are integrally formed with the receptacle 1 adjacent its inlet opening 6 or mouth 38 and are arranged to project from the receptacle 1 to a position alongside the collection bag 33. The embodiment shown in FIG. 1 shows flaps 10;10A that substantially run the length of the mouth 38 of the receptacle bag 1.

The securing means 11;11A are respectively fitted with peel away covers 12 & 12A which can be removed in order to activate the securing means. Once the securing means has been activated it can be used to secure the flap portions to the drain portion of the collection bag and to each other, thus forming a leak proof seal in this embodiment. Generally one flap portion is folded back, the collection bag is correctly positioned and then the second flap is secured to the collection bag.

The flap portions, once secured, prevent spillage while the collection bag is being emptied. They also act as a safety feature to minimise the likelihood that the drain 35 of the collection bag 33 will be displaced from the waste-material receiving chamber 5 during emptying of the collection bag 33. The securing means 11;11A keeps the inlet 6 of the receptacle in such a position so that the bag 1 is instantly ready for receiving the bodily waste from the collecting bag 33 once it has been configured to its discharge state.

The securing means 11 & 11A is strong enough to secure the receptacle to the collection bag and hold it in place while discharge occurs. The securing means system 10&10A, 11&11A and 12& 12A, are however detachably attachable to the collection bag as the disposable receptacle will be removed for disposal. Removal of a disposable receptacle from the collection bag may occur several times a day using a fresh disposable receptacle each time. Suitable securing means for use with any embodiment of the invention include: dispersal adhesives such those available from Henkel™ under the product name Adhesin J1125, J1610 or J1620; Hot. melt adhesives such as those available from Henkel™ under the product name Sanicare HM6410; and double sided adhesive tapes such as those available from 3M™ under product identifiers 9415, 9416 and 9425; and PPI type 228 available from PPI Adhesive Products Ltd, Waterford Industrial Estate, Cork Road, Waterford. A double-sided tape adhesive may also be employed. For example a double-sided tape with a permanent adhesive on one side and a non-permanent adhesive on the other can be employed. Suitable double-sided tapes include those with a relatively high-strength adhesive on one side and a relatively low-strength adhesive on the other. Desirably the relatively high-strength adhesive can be employed to attach the tape to the receptacle in a permanent fashion while the relatively low-strength adhesive allows the receptacle to be connected to the body of the collection bag or the drain thereof in a temporary (removable) arrangement. One or both sides of the tape can be provided with a peel-off protective layer to prevent undesired bonding. In one arrangement the tape is affixed to the disposable receptacle by the relatively high-strength adhesive while a peel-off protective layer is provided on the relatively low-strength adhesive. A user will then remove the peel-off protective layer to expose the relatively low-strength adhesive when it is desired to affix the disposable receptacle to the collection bag. Desirably the tape is also water-soluble. Optionally any peel-off layer is water-soluble. One commercially available tape is that sold under the trade name Mactac (for example Mactac 1121) MACtac UK, Northampton NN5 7UA, UK.

The receptacle 1 is desirably toilet-flushable to allow the receptacle to be disposed of conveniently. The disposable receptacle is preferably disposable by flushing it down a toilet. The weight of the waste material in the receiving chamber will assist in the flushing of the receptacle. The receptacle may also be disposed of by other means such as by placing in another container for example a “nappy sack”. The disposable receptacle may have an elongated body of which becomes narrower in a direction from the top end thereof towards the base end thereof. The tapered shape lends a flushable profile to the receptacle.

The desired way of disposing of a used receptacle is by flushing it down a toilet. The receptacle is preferably made of any suitable biodegradable material which can be disposed of by flushing in an environmentally friendly way. Desirably the receptacle is made of a cellulosic type material. Cellulosic fibres may be chemically treated either individually or the finished paper may be chemically treated to improve its wet strength or durability. The material of which the receptacle is constructed must have sufficient resilience so that it does not disintegrate when the waste material enters the receptacle. Suitable materials for manufacturing the receptacle include plastics films. Desirably the plastics film is water soluble. One such material is polyvinyl alcohol film which optionally is provided with a backing of non-woven water-soluble paper. The polyvinyl alcohol film may be surface treated to improve its wet strength and durability. Such treatments are well known to a person skilled in the art.

A water resistant paper such as ‘greaseproof’ paper may also be utilised. The materials may be from 26-32 gsm bleached, unbleached, colored greaseproof paper. Grease-proof paper may be obtained for example from Fort James Ireland Ltd, McKee Avenue, Finglas, Dublin 11 (Phone no +353 1 85032334) under the trade name Raytex Greaseproof Paper.

Examples of commercially available materials include MonoSol AF M1030 75 uM or 38 uM polyvinyl alcohol film sold by MonoSol, LLC 1701 County Line Road Portage, IN 46368 USA (European Agent Greensol 28 Rue des Grahuches Vauguillettes 89100 Sens France); MonoSol A127 50pM polyvinyl alcohol 5 film sold by MonoSol, LLC 1701 County Line Road Portage, IN 46368 USA (European Agent Greensol 28 Rue des Grahuches Vauguillettes 89100 Sens France; and Aicello Solublon PVAL sold by Syntaiia GmbH, Xantener St'r. 1, D-45479 Mulheim/Ruhr Germany films such as BP, LA and NP grades greaseproof for example as sold by Syntana GmbH, Xantener Str. I, D-45479 Mulheim/Ruhr Germany.

The receptacle is usually made from light deformable material that allows the device to be tucked away and stored discretely. The primary aspect of the present invention is that receptacle design and composition is conducive to providing a portable device. The light foldable material provides for a portable device that can be comfortably stored in a pocket or the like or, as with the preferred embodiments of the invention attached to the collection bag 33 to travel with the collection bag until such time as it is required for discharge use.

The receptacle may be provided with finger grip portions 7;7A. The finger grip portions 7;7A may project away from the receptacle body and the securing means. Preferably such finger gripping portions are positioned about the inlet opening 6 of the receptacle. The gripping portions 7;7A can be employed for the end user's convenience and to aid the user in disengaging of the securing means to allow the release of the disposable receptacle. The finger gripping portions 7;7A may be any size. Larger finger gripping tabs may be provided for patients who lack dexterity, such as elderly, patients with inflammation or chronic conditions such as arthritis, or disabled, in disengaging the securing means.

It is desirable that in some embodiments, the finger gripping portions 7;7A are distinguishable from the rest of the body to assist end users who have poor eye sight to locate it, For example, the finger gripping portion 7;7A may be a different colour to the rest of the body. Additionally or alternatively the finger gripping portions 7;7A may be a different texture to the rest of the body.

FIG. 1 further displays opposing pads 13 and 13A which act as wipe pads for the outlet 34 of the collecting bag and are also absorbent. FIG. 1 shows both pads arranged opposing each other on opposing sides of the receptacle. FIG. 1 shows the wiper pads extending a distance between 1 cm to 5 cm into the inlet opening of the bodily waste receiving chamber, most preferably the pads will extend any length between 1-2 cm into the inlet opening of the bodily waste receiving chamber. The pads 13;13A are arranged for wiping along the discharge outlet 34 of the collection bag 33. The pads 13;13A also serve to hold open an insertion portion 37 in the disposable receptacle 1 when it is attached to the collecting bag 33. This insertion portion 37 should be sufficiently wide enough to allow waste material to discharge into the disposable receptacle, and more particularly to allow ease of insertion of the drain 35 into the receptacle 1. The pads may be made of any adsorbent material. Most preferably the pads will be made from non woven cotton material or tissue paper.

The pads 13;13A serve three functions. A first function is to clean the drain of the collecting bag. They may additionally be used to absorb any residual bodily waste matter from the outlet 34 of the collection bag 33 (for example residual material present after emptying). They may also function as a visual marker identifying the correct position for the outlet 34 of the collection bag 33 relative to the receptacle 1.

The receptacle 1 for use in the collecting system desirably is self-securing to the collection bag 33 at a plurality of positions so as to ensure good retention of the receptacle 1 in the desired position.

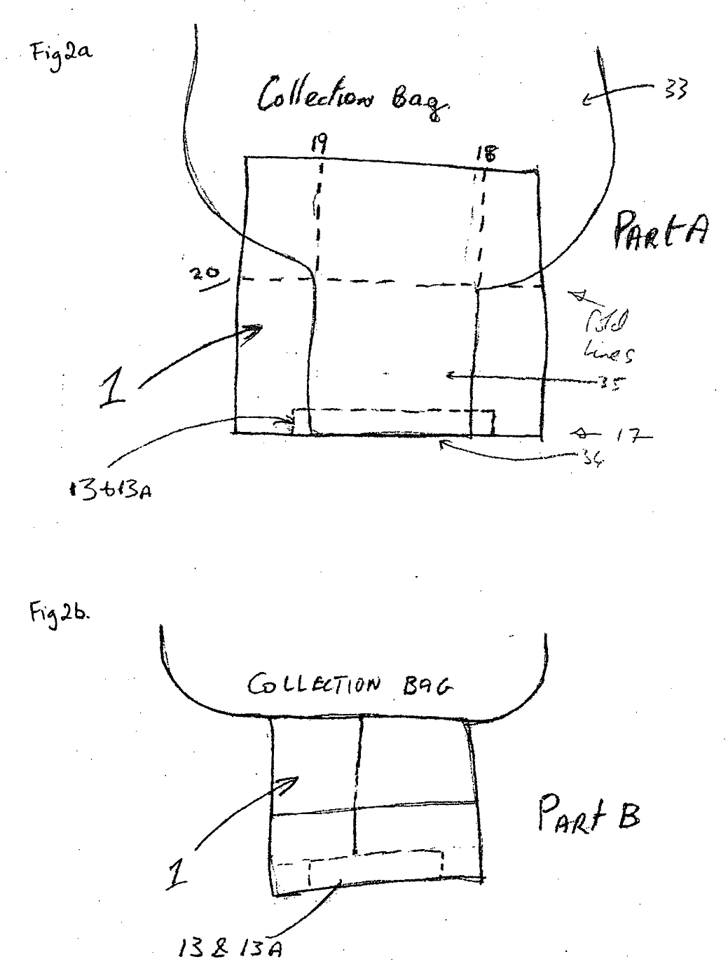

FIG. 2, in particular FIG. 2A shows the receptacle 1 folded toward the drain 35 of the collection bag along transverse (in the embodiment horizontal) fold line 17. Folding the bag in this way with the drain of the collection bag provides a restriction that prevents the discharge of bodily waste from the collection bag. FIG. 2A also displays a further transverse fold line 20 and two longitudinal (in the embodiment vertical) fold lines 18 and that illustrate one method of folding the receptacle 1 so as to configure the receptacle 1 and the bag 33 to a collecting state as shown in FIG. 3 (passing through the partially folded state of FIG. 2B). In general the receptacle 1 will generally have a transverse dimension (width) which is greater than that of the drain 35 of the collecting bag 33. Generally the sides of the receptacle 1 will be folded in (in this case by folding along fold lines 18, 19) and subsequently the bottom 4 of the receptacle 1 is folded up (in this case along fold lines 17 and 20). This results in the compact (folded) state of the receptacle as shown in FIG. 2. It also results in a restriction on the drain 35 as will be described in more detail below.

FIG. 3 shows the collecting system in its collecting state. The receptacle 1 is folded with the drain 35 of the collection bag 33 so that the two are folded together. It will be appreciated that folding twice or more in generally the same direction, causes a rolling effect (whether a flat roll or a rounded roll).

The bag 1 has been folded along fold line 18 and subsequently along fold line 19 and lastly along fold lines 17 and then 20. The bag 1 is then in a compact configuration. In particular it does not extend beyond the width of the collection bag 33 and more particularly the width of the collection bag drain 35. The compact nature of the receptacle allows the device to be conveniently placed in position for subsequent use and it remains on the collection bag while the collection bag collects material. The user is completely mobile, as the receptacle does not impose any restriction on movement unlike many prior art systems.

When the collecting system is in a collecting state as shown in FIG. 3 there is no communication of waste into the disposable receptacle. A restriction, in the embodiment formed by folds, including fold 17 at the drain 35 of the collecting bag 33, prevents the discharge of waste from the collecting bag into the disposable receptacle 1. In this state bodily waste may be collected from the body into the collecting bag. However even though the disposable receptacle is attached bodily waste will be restricted from flowing through to the disposable receptacle.

FIG. 3 shows the receptacle in a compact configuration. The disposable receptacle may be folded or rolled into the compact configuration. Any other suitable means may also be used to compact the disposable receptacle. The drain of the collecting bag may also be restricted by rolling both the receptacle and the drain of the collecting bag in the same roll.

The compact disposable receptacle may be held in place using a fastener 25 as shown in FIG. 3. The fastener will hold the collecting system in a collecting state by maintaining the disposable receptacle in the rolled or folded configuration. Preferably the fastener is a J clip component. Alternatively any adhesive fastener may be used or a mechanical fastener such as a hook and loop fastener may be used. In a particular embodiment the fastener may be adapted to cooperate with a fastener arrangement already on the collection bag.

The restriction may be removed by expanding/unfolding the compact disposable receptacle. The disposable receptacle may be expanded by unfolding or unrolling. Of course it will be necessary to unroll both the receptacle and the drain of the collecting bag if both have been rolled in the same roll, in order to remove the restriction.

Once the compact disposable receptacle has been expanded the collecting system is in a discharge state where material can be discharged from the collecting bag into the disposable receptacle via the collecting bag drain. This will automatically occur when the restriction is removed.

It will be appreciated that the disposable receptacle is attached to the collection bag when the collection bag is in an empty stage. The disposable receptacle can be attached before the collection bag is used for the first time. Once the disposable receptacle is used to discharge the bodily waste materials from the collection bag, a fresh disposable receptacle can be attached to be collection bag. The great convenience for users of the system is that there is no requirement (as with certain systems of the prior art) to position a collection container relative to the collection bag after the collection bag has been filled, and to discharge the contents without spillage. In such cases, the collection container is positioned relative to the collection bag only when the collection bag has collected bodily waste materials which it is desired to discharge.

In a further embodiment the collecting system described above may further comprise in a securing means for securing the material-receiving chamber relative to a disposal unit such as a sanitary ware item.

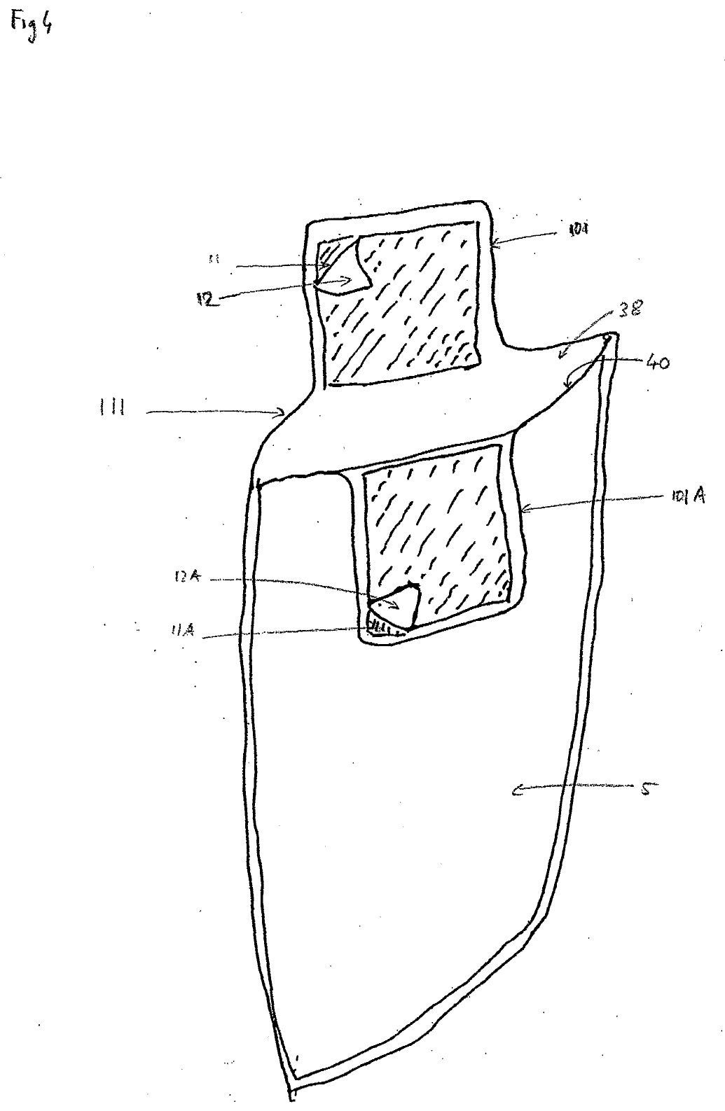

FIG. 4 shows an alternative embodiment for the receptacle 1 of the collecting system. Opposing flap portions 101 and 101A each integrally formed with opposing sides of the bag. On the respective flap portions 101;101A are located securing means 11 and 11A respectively. The receptacle comprises a mouth 38 that is formed by the upper periphery edge of the bag. The flap portions 101;101A are integrally formed with the receptacle 111 adjacent its mouth 38 and are arranged to project from the receptacle 111 to a position alongside a collection bag. The alternative embodiment shown in FIG. 1 shows flaps 101;101A that do not substantially run the length of the mouth 38 of the receptacle bag 1. The flaps are of sufficient dimensions to secure the receptacle to the collecting bag for discharge of materials but do not extend substantially beyond the drain of the collecting bag.

The securing means 11;11A are respectively fitted with peel away covers 12 & 12A which can be removed in order to activate the securing means. Once the securing means has been activated by removal of the covers, one and then the other can be used to secure the flap portions to opposing side of the drain portion of the collection bag as described above. The securing means 11;11A keeps the mouth 38 of the receptacle in such a position so that the bag 1 is in place and instantly ready for receiving the bodily waste from the collecting bag 33 once it has been configured to its discharge state.

Access to the disposable receptacle in the present embodiment is not restricted to an inlet as was the case in the first embodiment. The total width of the mouth 38 of the disposable receptacle 111 can be used to access the material receiving chamber 5. Therefore the present embodiment is suited for use with any sized collecting bag drain. It is desirable with such arrangements to provide an additional seal for sealing the mouth of the receptacle after discharge, as discussed below. Such a seal can be on the outside of the receptacle so that the top of the receptacle is rolled (folded) over upon itself and secured shut. The seal can be positioned below the mouth of the bag.

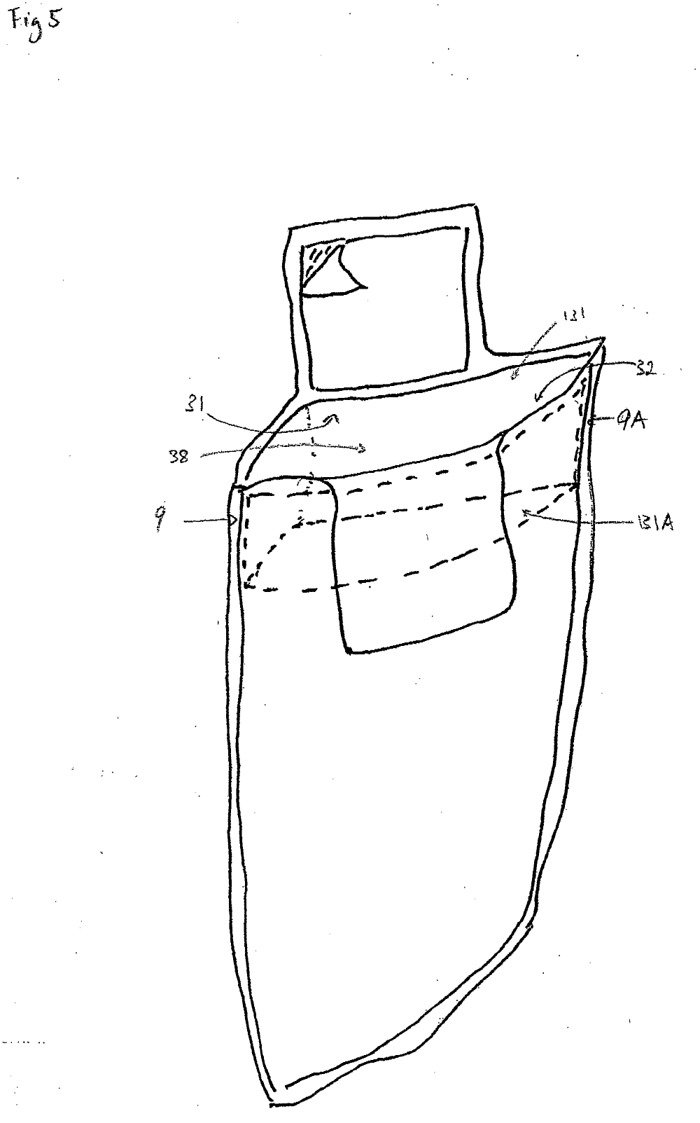

FIG. 5 shows the position of the wiper pads 131;131A in an embodiment of a receptacle. The wiper pads are positioned just below the mouth 38 of the bag and substantially extend the width of the mouth 38 of the disposable receptacle. The wiper pads extend from side wall 9 to side wall 9A. Wiper pads 131 and 131A may be secured at the side walls 9 and 9A and optionally the wiper pads 131 and 131A may also be secured to rear wall 31 and to front wall 32 respectively. One advantage of this particular embodiment is that wiper pads have an increased surface area on which absorption and wiping can take place. As with all embodiments it is desirable that the sidewall are expandable for example in a concertina-like fashion as described in PCT/IE2006/000064.

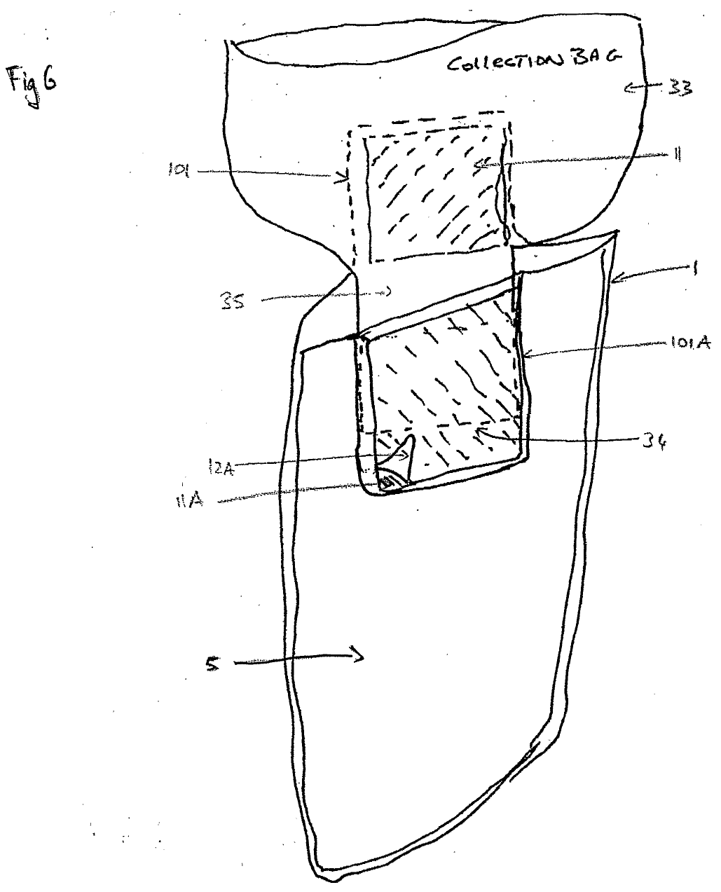

FIG. 6 shows the process of connecting of a receptacle to the collecting bag in a second embodiment of the collecting system.

The drain 35 of the collection bag extends through the mouth of the receptacle so that discharge outlet is contained in the material-receiving chamber 5.

The peel away cover 12 has been removed from securing means 11 so that the securing means 11 is activated. FIG. 6 shows flap 101 secured to the collecting bag using the activated securing means 11.

Flap 101A has yet to be attached to the collecting bag. The peel away cover 12A has not yet been removed. To ensure that the disposable receptacle 1 is securely attached to the collecting bag is will be necessary to remove the peel away cover and secure the second flap 101A to the collecting bag. Once both flaps have been secured to the collection bag the securing means 11 & 11A is strong enough to secure the receptacle to the collection bag and hold it in place while discharge occurs. The securing means system 10&10A, 11&11A and 12& 12A, are however detachably attachable to the collection bag as the disposable receptacle will be removed for disposal.

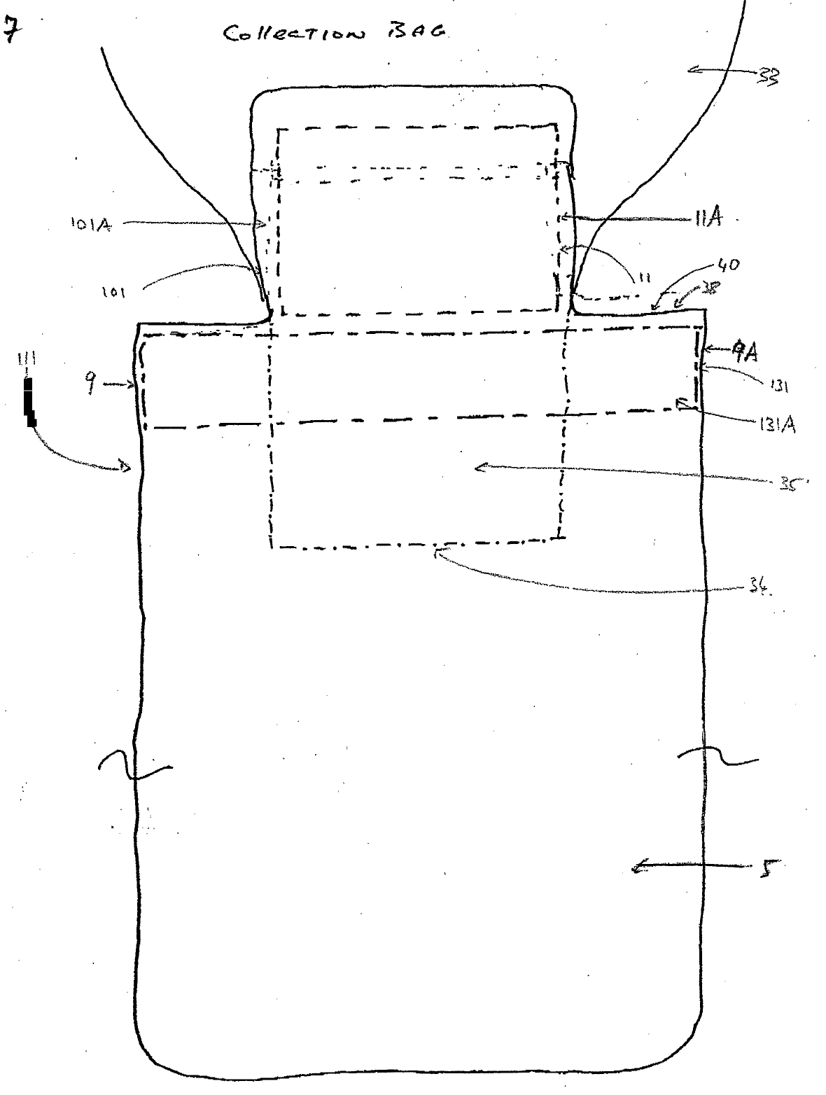

FIG. 7 shows the second embodiment of the collecting system in a discharge state. Both flaps 101 and 101A have been secured to the collecting bag using the activated securing means 11 and 11A. The drain of the collecting bag 35 extends into the material receiving chamber 5 and bodily waste material can be communicated from the collecting bag 33 to the disposable receptacle 111.

Wiper pads 131 and 131A are seen to extend the width of the receptacle mouth 38 and are positioned proximate to the peripheral edge 40 of the receptacle. When discharge has occurred the wiper pads are used to wipe as the receptacle is removed. The collection bag (now cleaned and empty) is ready to receive a fresh receptacle.

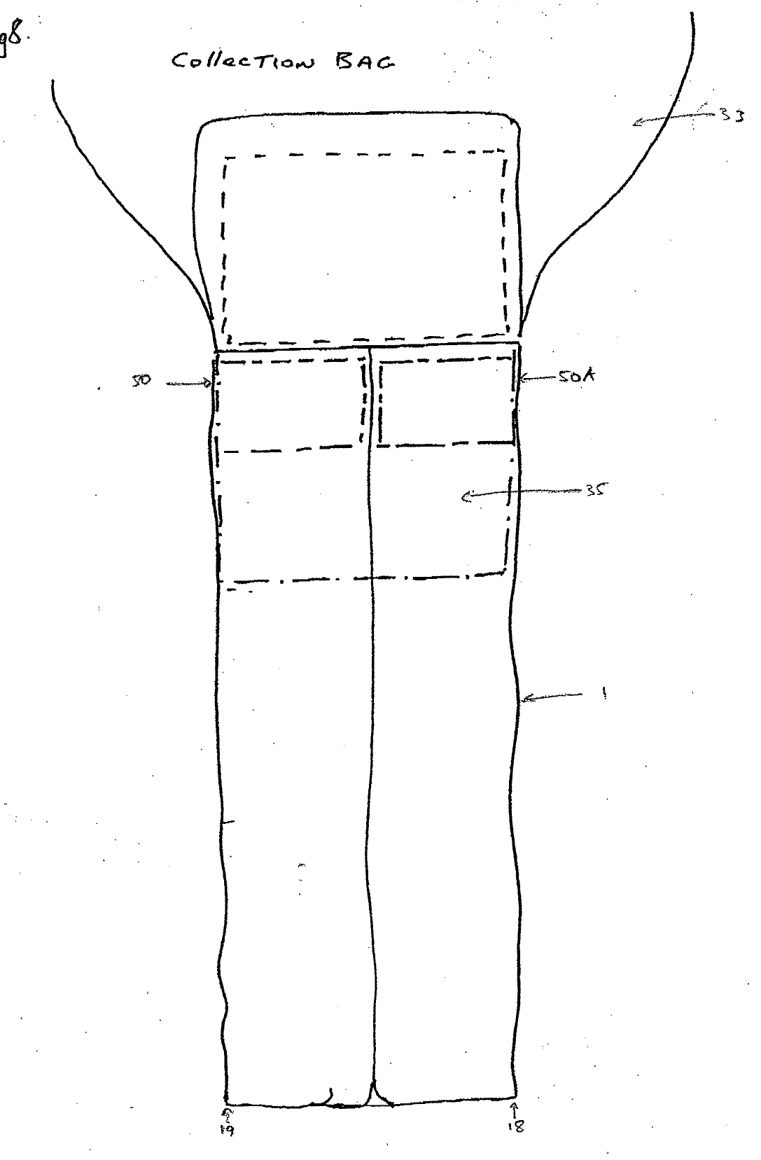

FIGS. 8,9 and 10 show how the bag 111 is moved from a discharge state where bodily waste is free to move from the collecting bag 33 into the collecting state where there is substantially no communication of waste. FIG. 8 shows the first step in moving the collecting system into a collecting state. FIG. 8 displays the bag 111 folded along fold lines 18 and 19. Fold lines 18 and 19 are positioned so that they are in line with the outer edges 50;50A of the collecting bag drain 35. Positioning the fold lines at outer edges 50 and 50A will ensure that the receptacle and the drain are substantially the same lateral dimensions and therefore can be easily rolled up together.

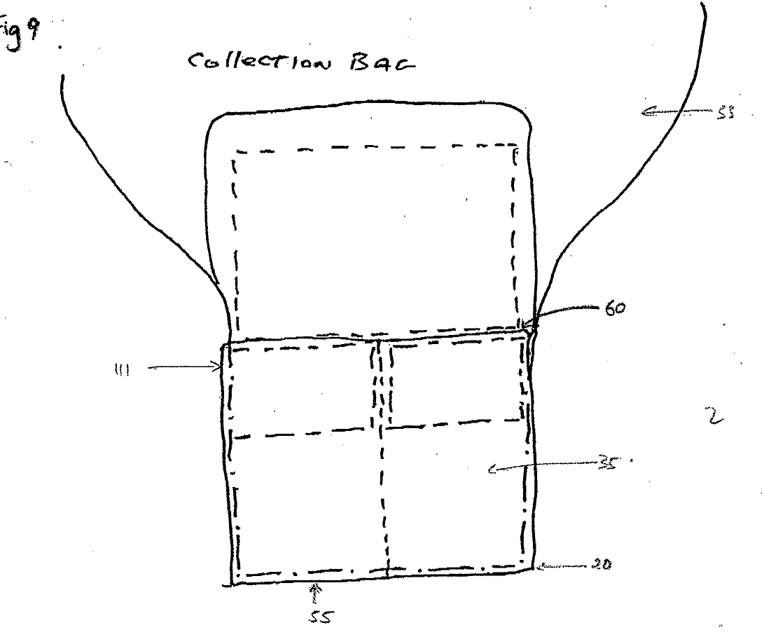

FIG. 9 shows a progression from FIG. 8 with a subsequent step involved in moving the collecting system into a collecting state. Once the disposable receptacle has been folded laterally the bag 111 is then folded transversely along fold line 20 to line up substantially with the end of the drain. Folding the bag 111 with the drain 35 of the collection bag 33 serves two functions (i) the bag becomes more compact and is more comfortable to be worn by the patient, the bag forms a compact structure that does not extend beyond the width of the collection bag discharge outlet 34 and (ii) a restriction 55 and a further restriction with each subsequent fold, is formed at the drain 35 of the collecting bag 33, which prevents the communication of bodily waste from the collection bag 33 into the disposable receptacle. This restriction 55 can be positioned further up the drain of the collecting bag 33 by further folding the folded receptacle and drain 35 towards the top end 60 of the drain 35.

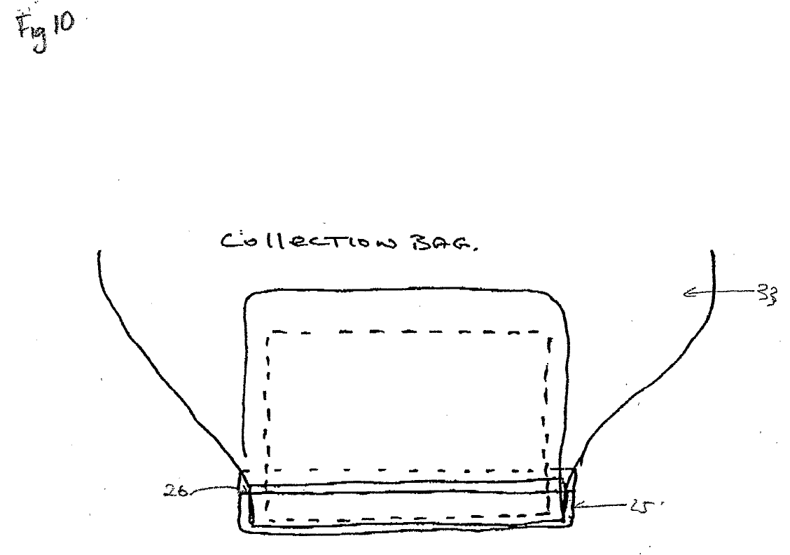

FIG. 10 shows the collecting system in a collecting state wherein the restriction 55 has been positioned further up the drain of the collecting bag 33 by further folding the folded receptacle and drain 35 to the top end 60 of the drain 35.

The collecting system as shown in FIG. 10 has been folded into a more compact orientation. Folding the bag 111 beyond the discharge outlet 34 of the collecting bag 33 not only restricts the passage of waste material into the material receiving chamber 5 of the receptacle 111 but also restricts the passage of material through the drain 35 of the collecting bag 111. It should also be remembered that the act of folding the receptacle along the fold lines and/or folding it within the drain assists in the sealing process.

The bag is secured by a plastic J clip 25 (see FIG. 3) which acts as an independent fastener to maintain the folded receptacle and folded drain in the folded position. The bag may be secured using any suitable fastener. The fastener provides a secondary closure. The fastener or J clip 25 may be fitted with a hook and loop fastener attachment 26 or similar attachment to mate with any additional sealing arrangement which exists on the collection bag. This gives additional security to the end user that there will be no accidental spillage from the collection bag and bodily waste receiving chamber combination.

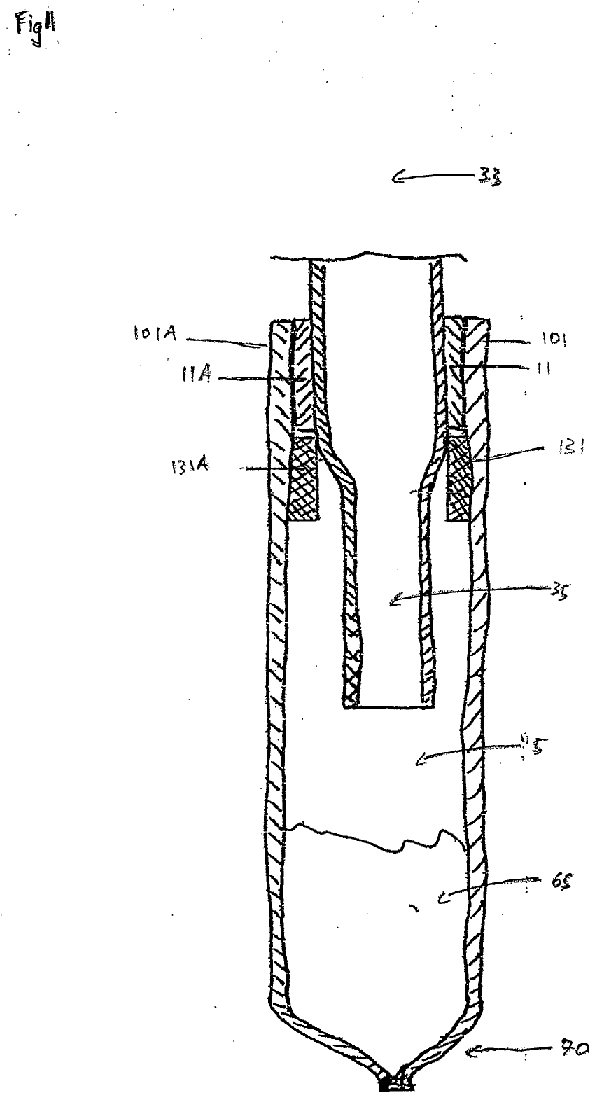

FIG. 11 shows a cross sectional view of the collecting system in the discharge state, where bodily waste 65 has been discharged from the collecting bag 33 through the drain 35 into the waste receiving chamber 5. The flaps 101 and 101A hold the disposable receptacle in position during and after discharge of the bodily waste material using the securing means 11 and 11A. FIG. 11 also shows the wiper pads 131 and 131A flanked on either side of the collecting bag drain 35 so that they will readily absorb any residual bodily waste on removal of the disposable receptacle 111. The disposable receptacle 111 can be disposed of once it has been removed from the collecting bag 33. The preferred method of disposal is by flushing down the toilet. The tapered end profile 70 of the receptacle will aid in the disposal of the receptacle.

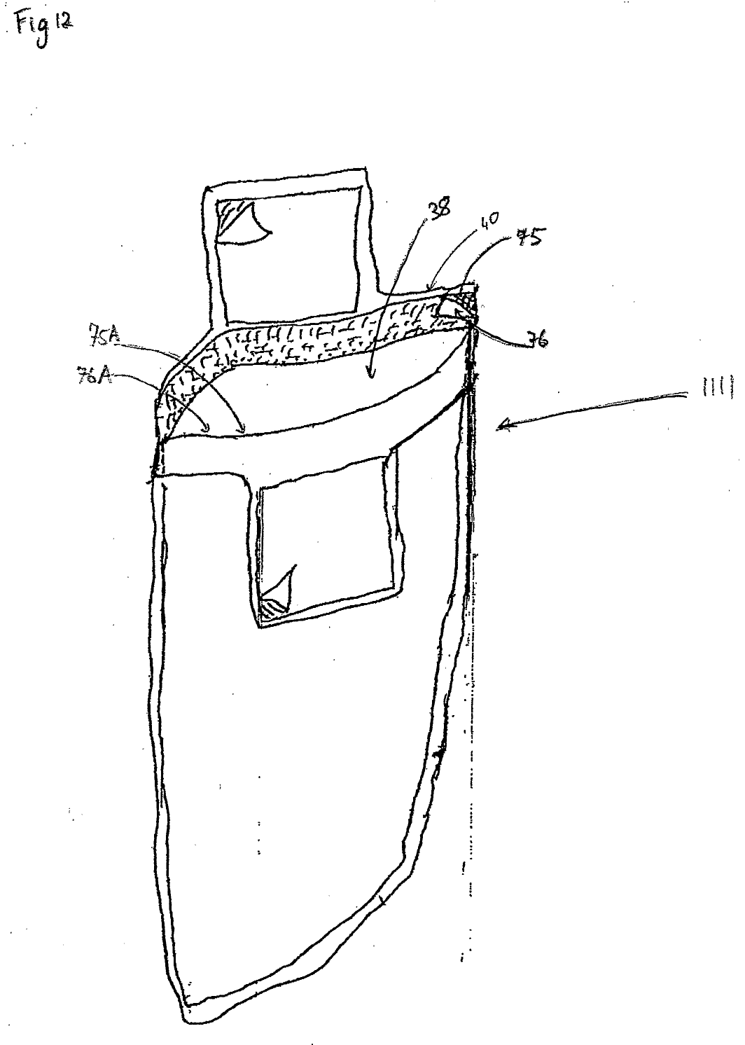

FIG. 12 shows the receptacle component 1111 of the collecting system provided with sealing means separate from the flaps for closing the receptacle. The sealing means is preferably independent strips of adhesive 75 and 75A that are located at the mouth 38 of the receptacle or positioned proximate to the peripheral edge 40 of the receptacle 1111.

The strips 75 and 75A can be used for closing the mouth 38 of the disposable receptacle once the receptacle containing discharged bodily waste material has been removed from the collecting bag.

Each of the strips 75 and 75A is respectively fitted with peel away covers 76 & 76A which can be removed in order to activate the sealing means. Once both the sealing means have been activated they can be used to seal the mouth 38 of the receptacle, thus forming a leak proof seal. Both strips 75 and 75A engage each other to form the leak-proof seal.

Sealing the mouth 38 of the disposable receptacle 111 will prevent undesired spillage of the waste material once the receptacle, containing discharged bodily waste, has been removed for disposal. The seal also allows the receptacle to be disposed into a waste bin rather then flushed down the toilet.

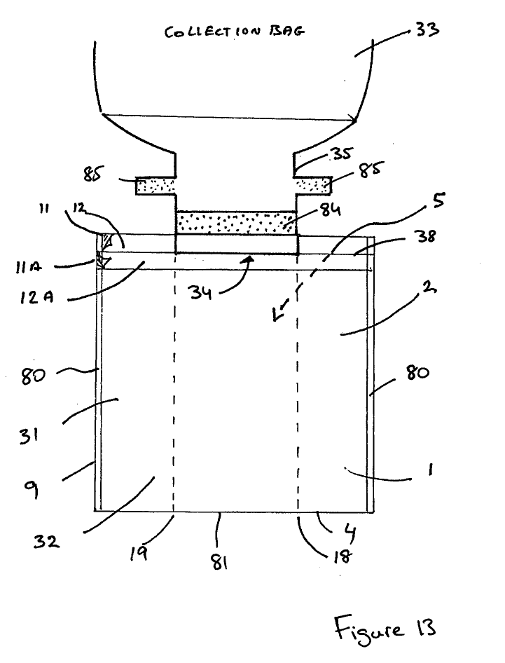

FIG. 13 shows a further embodiment which is similar to that of FIG. 1 and the same reference numerals have been used for like parts.

In particular the embodiment of FIG. 13 shows a specific desirable embodiment of the receptacle 1. In the embodiment the receptacle 1 is made from a water-soluble plastics material. This material is sufficiently water-resistant to maintain its integrity when holding or receiving the contents of the collection bag (at least during the discharge process) but sufficiently soluble to make it toilet-flushable. The receptacle is formed by folding a piece of plastic upon itself and welding closed the sides to form a bag with the fold forming the base of the bag. The figure shows the fold 81 and the welded seams 80 along the side.

In this simple construction there are no pads as appeared in other embodiments.

As is further desirable (in particular in combination with use of a water soluble plastics material) the adhesive strips 11 and 11A are formed by a double-sided adhesive tape. The tape is more strongly adhered to the receptacle 1 than it is adhered to the collection bag 33. As stated above this can be achieved using an adhesive which forms a different strength of bond on either side. In general the adhesive which is used to affix the receptacle 1 to the collection bag 33 forms a temporary bond which allows the receptacle 1 to be detached from the collection bag 33 when desired.

FIG. 13 also demonstrates the use of a collection bag which has a fastener arrangement already on the collection bag 33. In particular the bag 33 has a hook and loop fastener arrangement which is generally used for closing the collection bag. The fasteners take the form of a main pad 84 which runs across the entire width of the drain 35 and adjacent the outlet thereof. Two arms or tabs 85 are extend out from opposing sides of the drain 35 at a higher up position form the other part of the fastening arrangement. In particular when the collection bag is in normal use (without any receptacle 1 attached) the drain 35 is folded (rolled) up the arms 85 fasten to the pad 84 closing the drain 35 by holding the drain in the folded up configuration and by forming a restriction across the drain. Generally the arms 85 and the pad 84 will be a hook and loop fastener arrangement.

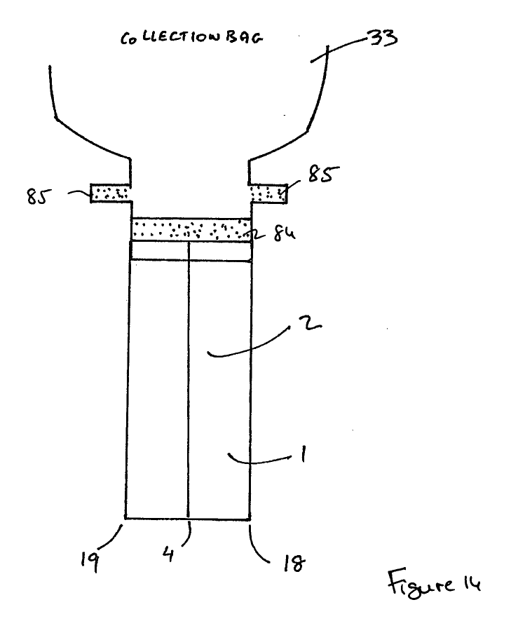

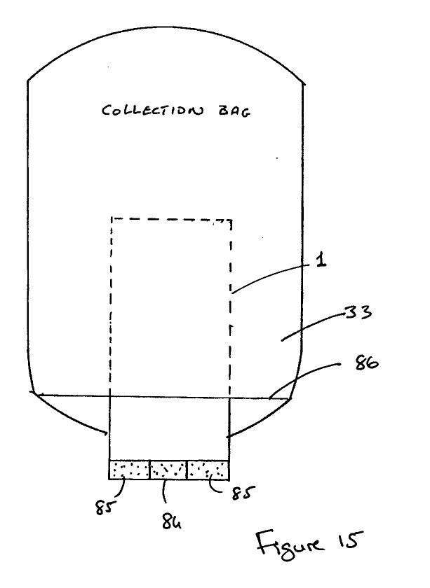

In the embodiment the receptacle 1 is folded up, firstly to the position shown in FIG. 14 (and in a manner analogous to that shown for the embodiment of FIG. 1) by folding about fold lines 18,19, and then to the position shown in FIG. 15. In FIG. 15 the arms 85, have been folded across onto the pad 84 so as to interlock the arms 85 and the pad. This holds the receptacle 1 in position connected to the collection bag 33. In both a collection state and a discharge state the discharge outlet of the collection bag is connected to the inlet opening of the disposable receptacle. As shown in FIG. 15, if the collection bag is provided with a pocket 86, the receptacle 1 can be inserted into the pocket 86 so as to hold it in the desired position. The collection bag 33 can then be used as normal by a wearer. When it is time to empty the collection bag, the arms 85 are separated from the pad 84, and the receptacle 1 is moved back into the position of FIG. 14 and then FIG. 13 so that it can receive the contents of the collection bag. Such discharge from the collection bag is automatic because the restriction is removed from between the collection bag and the reservoir.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1-61. (canceled)

62. A collecting system comprising a
(i) collecting bag for collecting bodily waste materials, the collecting bag comprising a discharge outlet;
(ii) a disposable receptacle for receiving bodily waste materials from a discharge outlet of the collecting bag, the receptacle comprising an inlet opening formed on a receptacle body; and
(iii) a securing means for securing the receptacle to the collecting bag,
the disposable receptacle and collecting bag being movable between
(a) a collecting state where the collecting bag is arranged for collecting bodily waste materials and there is substantially no communication of bodily waste materials into the disposable receptacle; and
(b) a discharge state where bodily waste materials can be discharged into the disposable receptacle,
and in both states the discharge outlet of the collection bag is connected to the inlet opening of the disposable receptacle.

63. The collecting system of claim 62, wherein a restriction of the discharge outlet prevents the flow of waste into the disposable receptacle.

64. The collecting system of claim 63, wherein the restriction is formed by rolling the receptacle.

65. The collecting system of claim 63, wherein the restriction is formed by rolling both the receptacle and the discharge outlet of the collecting bag in the same roll.

66. The collecting system of claim 63, wherein the contents of the collecting bag can flow into the receptacle once the restriction on the discharge outlet has been removed.

67. The collecting system of claim 62, wherein the receptacle body is adapted to receive within the inlet opening the bodily waste discharge outlet of the collection bag.

68. The collecting system of claim 62, wherein the receptacle comprises a bodily waste material-receiving chamber and the securing means holds the bodily waste material-receiving chamber in position for discharge of the bodily waste materials into the bodily waste material-receiving chamber from the collection bag.

69. The collecting system of claim 62, wherein the receptacle is formed from a plastics material that disintegrates in water.

70. The collecting system of claim 69, wherein the plastics material is a polyvinyl alcohol material.

71. The collection system of claim 62, wherein the securing means comprises double-sided adhesive tape.

72. The collecting system of claim 62, further comprising a fastener wherein the fastener is used to hold the system in a collecting state.

73. The collecting system of claim 62, wherein the receptacle further comprises a guard portion which is attached to the receptacle adjacent the inlet opening and which is arranged to project from the receptacle to a position alongside the collection bag.

74. The collecting system of claim 62, wherein the receptacle is toilet-flushable.

75. The collecting system of claim 62, further comprising at least one finger grip portion which projects away from the receptacle body about the inlet opening.

76. A method for collecting bodily waste and disposing of the bodily waste materials comprising the steps of:
(i) attaching a disposable receptacle with an inlet opening, to a collecting bag with a bodily waste discharge outlet, so that the inlet opening receives the bodily waste discharge outlet of the collection bag;
(ii) arranging the disposable receptacle and collecting bag into a collecting state where the collecting bag is arranged for collecting and there is no communication of waste into the disposable receptacle;
(iii) collecting bodily waste from the body into the collecting bag;
(iv) moving the disposable receptacle into a discharge state wherein the bodily waste material can be discharged from the collecting bag into the disposable receptacle;
(v) discharging the bodily waste materials into the receptacle; and
(vi) optionally disposing of the receptacle and any contents thereof.

77. A disposable receptacle for receiving bodily waste materials from a discharge outlet of a collecting bag, the receptacle comprising:
(i) a bag body having front and rear walls and a mouth; and
(ii) securing means in the form of a double-sided adhesive tape one side of which is attached to the receptacle and the other side is provided with a protective layer which is removable for adhesively securing the receptacle to the collection bag.

78. The disposable receptacle of claim 77, wherein the bag body is constructed of a plastics material which disintegrates in water.

79. The disposable receptacle of claim 78, wherein the plastics material is a polyvinyl alcohol material.

80. A disposable receptacle for receiving bodily waste materials from a discharge outlet of a collecting bag, the receptacle comprising:
(i) a bag body having front and rear walls and a mouth;
(ii) a front securing flap integrally formed with the front wall of the bag and arranged to project above the mouth of the bag and a rear securing flap integrally formed with the rear wall of the bag and arranged to project above the mouth of the bag; and
(iii) securing means on each of the flaps for securing the flaps to opposing sides of the collecting bag,
the flaps, in use, for securing the receptacle to a collecting bag.

81. A disposable receptacle for use in a collecting system according to claim 62.
